(19) United States
(12) Patent Application Publication
(10) Pub. No.: US 2008/0132751 A1
(43) Pub. Date: Jun. 5, 2008

(54) Compositions and methods for treatment of pain

(75) Inventors: Brian C. Keller, Antioch, CA (US); Camilo Rey, Weston, FL (US); Jorge Useche, Bogota (CO)

Correspondence Address: THELEN REID BROWN RAYSMAN & STEINER LLP, P. O. BOX 640640, SAN JOSE, CA, 95164-0640, US

(73) Assignee: BetaZone Laboratories, Inc.

(21) Appl. No.: 11/607,830

(22) Filed: Nov. 30, 2006

(51) Int. Cl. A61M 21/02; C07C 59/00; C07D 489/00

(52) U.S. Cl. 600/26; 546/237; 546/44; 546/74; 554/213

(57) ABSTRACT

The present invention provides compositions and methods for treating pain by the administration of analgesics. The analgesics are encapsulated in diacylglycerol-polyethyleneglycol (DAG-PEG) liposomes and delivered sublingually as aerosols.

### FIELD OF THE INVENTION

This invention relates to the administration of oral analgesics by spraying methods and compositions. The invention specifically relates to formulations and methods for the delivery of liposomal analgesic aerosols via sublingual administration.

### BACKGROUND OF THE INVENTION

Pain is the result of the perturbation of a complex set of neurobiological mechanisms manifesting a response in the patient consisting of discomfort, agitation, nausea, vomiting, psychosomatic changes and even suicide ideations. A primary goal in treating a patient in pain is the speed of onset of the given remedy. Analgesics have, for thousands of years, been administered by the oral route of administration in dosage forms such as liquids, tablets and capsules. The therapeutic effect of a drug is directly related to the quantity and rate at which the unchanged drug reaches the bloodstream. For many analgesic drugs the formulation and the route of administration have a great effect on both of these parameters.

Oral administration with subsequent swallowing of the analgesic preparation presents specific problems once the drug is in the lower GI tract. The low pH of the stomach (pH 1-2) can have a destructive effect on a drug, rendering it un-usable, un-absorbable, less potent or completely inactive. Direct drug uptake at any point along the GI tract shunts the drug into the hepatic portal venous system and into the liver where the liver begins to metabolize the drug in an attempt to make it water soluble for excretion. This is known as the first-pass effect which can prevent part, or all, of the drug from entering the bloodstream and negating its therapeutic effect. This process, accompanied with the dissolution of a solid dosage form takes time, which will delay the onset of action of the drug and subsequently the relief of pain.

With the rare exception of uptake of large particles (up to several micrometers), which occurs through M cells on Peyer's patches, molecule are absorbed only when they are dissolved. Absorption therefore depends on their solubility in the dosage form in which it is delivered, or in the environment to which it is delivered, and the lipophyllic character of the drug substance itself.

The mucosa of the mouth and throat is highly vascularized and ideal for the absorption of drugs in the inner oral cavity. This route of administration is particularly advantageous for compounds that are needed to have a rapid onset of action or are not well absorbed when take orally. This route of administration circumvents exposure of compounds to digestive enzymes and the high acidity of the GI tract and further avoids the first-pass effect. One example of inner-oral delivery, specifically, sublingual delivery is nitroglycerin. By placing nitroglycerin tablets or liquid under the tongue rapid onset is achieved by virtue of quick absorption into the blood stream through the two lymphatic ducts located under the tongue. This route avoids the liver where the compound is highly metabolized on the first exposure to metabolic enzymes. The mechanism by which inter-oral absorption takes place is by passive diffusion. Absorption of molecules through oral mucosa depends therefore on their lipophilicity.

The term bioavailability is a term used for the clinical description of the completeness of absorption in vivo and indicates the extent to which a substance reaches the bloodstream. It is defined as the fraction or percentage of the administered dose that is ultimately absorbed intact. Consequently, if a drug is made more soluble or more lipophyllic, or both, the bioavailability can be enhanced. This is particularly true with drugs that are intended to be absorbed through an inner-oral route.

Liposomes have been shown to increase bioavailability of some drugs, e.g. CoEnzyme Q10 (ubiquinone—a treatment for congestive heart failure) and melatonin. (Trends in Food Science & Technology, B Keller, Liposomes in Nutrition, Chapter 12, Elsevier, 2001.) In a randomized double-blind study, each group was given one dose of either sublingual liposome-encapsulated drug or an oral two-piece gelatin capsule. The liposomal sublingual formulation provided better bioavailability for both drugs. Both liposomal formulations used dipalmitoylphosphatidylcholine (DPPC) and required vortexing.

### BRIEF DESCRIPTION OF THE INVENTION

The present invention provides compositions and methods for treating pain by the administration of analgesics. The analgesics are encapsulated in diacylglycerol-polyethyleneglycol (DAG-PEG) liposomes and delivered sublingually as aerosols.

### DEFINITIONS

“Diacylglycerol-polyethyleneglycol (DAG-PEG)” refers to a lipid with a three-carbon-chain backbone and having acyl groups attached to two of the three carbons and a polyethylene chain attached to the other carbon. The acyl and polyethylene glycol chains may be attached to the backbone by a variety of chemical linkages, including but not limited to, ester and ether bonds. Linkers may be provided between the backbone and the chains. The chains may be attached at any position of the backbone.

“Aerosol” refers to a fine mist or spray which contains minute particles.

“Aerosol deliverer” refers to a device for converting a liposome suspension into an aerosol and delivering the aerosol to a patient. The aerosol deliverer typically is provided with a reservoir for containing the liposome suspension prior to delivery. Aerosol delivers include mechanical and electrical pumps, misters, nebulizers, and the like.

### DETAILED DESCRIPTION OF THE PREFERRED EMBODIMENTS

Liposome technology has been known to enhance uptake, or facilitate delivery of various drugs. For example, the parenteral and topical uses of liposomal carriers will protect a drug against the hostile biological milieu and provide increased penetration into tissues, the intended site for drug action and pharmacology. Encapsulating a drug into a lipid carrier like a liposome has specific advantages for enhancing the effect and outcome of a drug therapy. Encapsulating an oral analgesic into a liposome and administering the resultant preparation into the mouth the lipid carrier allows better inner oral penetration of the drug/liposome complex and subsequently relieves pain faster due to direct entry into the bloodstream, avoiding the lower GI tract, the hepatic-portal system and the first-pass metabolic loss of drug. This process will directly increase the drugs bioavailability.

The present invention provides compositions and methods for the delivery of pain-relieving drugs pain such as non-opiate analgesic agents, (phenacetin, and acetaminophen), nonsteroidal anti-inflammatory agents (diclofenac, ibuprofen, naproxen, and ketoprofen), aspirin and its derivatives, and narcotic analgesics (morphine, butorphanol). Because liposomes have both an aqueous internal space as well a lipid bilayer, virtually any such drug can be encapsulated to some extent.

Liposomes comprised of diacylglycerol-polyethyleneglycol (DAG-PEG) have been disclosed in co-owned U.S. Pat. No. 6,610,322, which is hereby incorporated by reference, and in U.S. Pat. No. 6,958,160, which is hereby incorporated by reference. It has been discovered that DAG-PEG liposomes are especially suitable for sublingual delivery of analgesics via an aerosol spray. The present invention takes advantage of properties inherent in DAG-PEG liposomes, such as stability and ease of formulation.

An advantage of the present invention is the ease of formulations provided by the DAG-PEG lipids. DAG-PEG lipids allow spontaneous liposome formation at low temperatures without the need for further liposome sizing. Additional lipid and non-lipid components can easily be incorporated into the liposome formulation. Such additional components may include flavor enhancers (e.g., mint, sugar, sucralose, etc.), preservatives (e.g., ethyl alcohol, glycerin, potassium hydroxide), and mouth feel enhancers (e.g. sorbitol, sodium citrate, PVP).

Another advantage is the increased bioavailability provided by administering the formulations sublingually. When the formulations of the present invention are administered sublingually the bioavailability of the analgesic, measured at fifteen minutes after administration, is preferably greater than about 25 percent better than that achieved by oral administration. More preferably, the bioavailability is greater than about 50 percent better. Most preferably, the bioavailability is greater than about 100 percent better. Also, when the formulations of the present invention are administered sublingually the bioavailability of the analgesic, measured at thirty minutes after administration, is preferably greater than about 25 percent better than that achieved by oral administration. More preferably, the bioavailability is greater than about 50 percent better. Most preferably, the bioavailability is greater than about 100 percent better.

Though administration by aerosol spray is the preferred method in practicing this invention, sublingual delivery of the liposomal formulations is also effective.

In a preferred embodiment, the invention comprises a method of delivering an analgesic by combining the analgesic with a DAG-PEG to produce a liposome suspension; and administering the suspension sublingually. The suspension is delivered by means of an aerosol spray. The analgesic may be chosen from the group comprising naproxen, ibuprofen and acetaminophen ketoprofen, diclofenac, hydrocodone, morphine, fentanyl, hydromorphone, methadone, meperidine, oxycodone, and levorphanol. The method may include providing the liposome suspension in the reservoir of an aerosol deliverer. The DAG-PEG has a P.sub.a between about 0.84 and 0.88 and a P.sub.v between about 0.88 and 0.93 and where P.sub.a is the packing parameter with respect to surface and P.sub.v is the packing parameter with respect to volume. The combining occurs at a temperature above the melting point of the DAG-PEG. The PEG chain of the DAG-PEG preferably has a molecular weight between about 300 Daltons and 5000 Daltons. The DAG-PEG may be selected from the group consisting of PEG-12 glycerol dioleate (GDO), PEG-12 glycerol dimyristate (GDM), PEG-23 glycerol dipalmitate (GDP), PEG-12 glycerol distearate (GDS), and PEG-23 GDS, where the number after “PEG” indicates the numbers of C₂H₄O subunits in the PEG chain. The melting point of the DAG-PEG is preferably below about 40 degrees C., and the acyl chains of the DAG-PEG are preferably greater than or equal to 14 carbons in length.

In another preferred embodiment the invention comprises an aerosol delivery system having an aerosol deliverer; a reservoir; and a liposomal suspension contained in the reservoir, where the liposomal suspension comprises an analgesic and a DAG-PEG. The analgesic may be chosen from the group comprising naproxen, ibuprofen and acetaminophen, ketoprofen, diclofenac, hydrocodone, morphine, fentanyl, hydromorphone, methadone, meperidine, oxycodone, and levorphanol. The DAG-PEG may have a P.sub.a between about 0.84 and 0.88 and a P.sub.v between about 0.88 and 0.93 and where P.sub.a is the packing parameter with respect to surface and P.sub.v is the packing parameter with respect to volume. The PEG chain of the DAG-PEG may have a molecular weight between about 300 Daltons and 5000 Daltons. The DAG-PEG may be selected from the group consisting of PEG-12 glycerol dioleate (GDO), PEG-12 glycerol dimyristate (GDM), PEG-23 glycerol dipalmitate (GDP), PEG-12 glycerol distearate (GDS), and PEG-23 GDS, where the number after “PEG” indicates the numbers of C₂H₄O subunits in the PEG chain. The melting point of the DAG-PEG is preferably below about 40 degrees C., and the acyl chains of the DAG-PEG are greater than or equal to 14 carbons in length.

In still another preferred embodiment the invention comprises a liposomal suspension including a DAG-PEG; and an analgesic. The analgesic may be chosen from the group comprising naproxen, ibuprofen and acetaminophen, ketoprofen, diclofenac, hydrocodone, morphine, fentanyl, hydromorphone, methadone, meperidine, oxycodone, and levorphanol. The DAG-PEG may have a P.sub.a between about 0.84 and 0.88 and a P.sub.v between about 0.88 and 0.93 and where P.sub.a is the packing parameter with respect to surface and P.sub.v is the packing parameter with respect to volume. The PEG chain of the DAG-PEG may have a molecular weight between about 300 Daltons and 5000 Daltons. The DAG-PEG may be selected from the group consisting of PEG-12 glycerol dioleate (GDO), PEG-12 glycerol dimyristate (GDM), PEG-23 glycerol dipalmitate (GDP), PEG-12 glycerol distearate (GDS), and PEG-23 GDS, where the number after “PEG” indicates the numbers of C₂H₄O subunits in the PEG chain. The melting point of the DAG-PEG is preferably below about 40 degrees C., and the acyl chains of the DAG-PEG are greater than or equal to 14 carbons in length.

### EXAMPLE 1

### Acetaminophen Liposomes

In a vessel, APAP was dissolved in PEG-12 Glycerol Dioleate and heat to 35 degrees C. while stirring slowly. 30% of the total water concentration was added to allow vesicle formation. Stirring continued for 5 minutes. Glycerin and propylene glycol and PEG-400 were added while stirring. In a separate vessel PEG 1450 was melted by heating to 40° C. and then mixed slowly. PVP K 29/32 and remaining water was added to step 4 while stirring. The contents of both vessels were co-mingled while mixing at moderate speed. Mixing continued for 5 minutes. Separately sucralose, sodium citrate, acesulfame K, and mint were added and mixed for 10 minutes. Formula was cooled to room temperature. The presence of liposomes was determined by using light a microscope with optical polarizer at 800×. Liposomes appeared as distinct round, silver bodies with a hair line cross like structure criss-crossing the entire vesicle.

Product: Acetaminophen Adults LipoSpray

| | Acetaminophen |
|---|---|
| | Adults LipoSpray |
| Acetaminophen (APAP) | 12% |
| Citric Acid | 0.5% |
| Sodium Citrate | 0.6% |
| Propylene glycol | 25% |
| Polyethylene glycol | 10% |
| 1450 | |
| Polyethylene glycol 400 | 10% |
| Sucralose | 0.5% |
| Acesulfame K | 0.4% |
| PVP K 29/32 | 0.5% |
| PEG-12 Glycerol | 4% |
| Dioleate | |
| Mint | 0.1% |
| Glycerin | 7% |
| Polysorbate 20 | 2% |
| Water CSP | 100% |

### EXAMPLE 2

### Ibuprofen Liposomes

Liposomes were prepared similarly to the method of example 1.

Product: Ibuprofen LipoSpray

| | Ibuprofen LipoSpray |
|---|---|
| Ibuprofen | 14.3% |
| Potassium Hydroxide | 7% |
| Sodium Citrate | 0.6% |
| Propylene Glycol | 5% |
| Polyethylene glycol | 12% |
| Caprylic/Capric | 25% |
| triglycerides | |
| Sucralose | 0.5% |
| Acesulfame-K | 0.4% |
| PVP K 29/32 | 0.5% |
| PEG-12 Glycerol | 7% |
| Dioleate | |
| Glycerin | 10% |
| Menthol | 0.3% |
| Mint | 0.3% |
| Polysorbate 20 | 2% |
| Water qs ad | 100% |

### EXAMPLE 3

### Naproxen Liposomes

Liposomes were prepared similarly to the method of example 1.

Product: Naproxen LipoSpray

| | Naproxen LipoSpray |
|---|---|
| Sodium Naproxen | 7% |
| Sorbitol | 10% |
| GDO 12 | 3% |
| Ethyl Alcohol | 5% |
| Menthol | 0.1% |
| Cinnamon Flavor | 0.1% |
| Acesulfame K | 0.5% |
| Water qs ad | 100% |

### EXAMPLE 4

### Ketoprofen Liposomes

Liposomes were prepared similarly to the method of example 1.

Product: Ketoprofen LipoSpray

| | Ketoprofen LipoSpray |
|---|---|
| Ketoprofen | 7% |
| Sorbitol | 10% |
| PEG-12 Glycerol | 4% |
| Dioleate | |
| Ethyl Alcohol | 7% |
| Menthol | 0.1% |
| Cinnamon Flavor | 0.1% |
| Acesulfame K | 0.5% |
| Water qs da | 100% |

### EXAMPLE 5

### Diclofenac Liposomes

Liposomes were prepared similarly to the method of example 1.

Product: Diclofenac LipoSpray

| | Diclofenac LipoSpray |
|---|---|
| Potassium Diclofenac | 3.6% |
| Silica Dioxide | 0.5% |
| Sodium Chloride | 1.6% |
| Povidone | 1.5% |
| Sodium Benzoate | 0.1% |
| L-menthol | 0.001% |
| 70% sorbitol solution | 0.08% |
| Citric acid | 0.6% |
| Sodium saccaharin | 0.3% |
| 30% Simethicone | 0.1% |
| Solution | |
| Ethyl Alcohol | 5% |
| Strawberry Flavor | 0.5% |
| PEG-12 Glycerol | 4% |
| Disterate | |
| Purified Water | 100% |

### EXAMPLE 6

### Bioavailability of Ibuprofen and Acetaminophen Liposomes in an Animal Model

In randomized double blind experiments, bioavailability of sublingual aerosol liposome formulations is compared to standard oral administration. In an animal model, sublingual liposomal administration provides superior bioavailabilty for both ibuprofen and acetaminophen.

### EXAMPLE 7

### Bioavailability of Ibuprofen and Acetaminophen Liposomes in Humans

In randomized double blind experiments, bioavailability of sublingual aerosol liposome formulations is compared to standard oral administration. In humans, sublingual liposomal administration provides superior bioavailabilty for both ibuprofen and acetaminophen.

What is claimed is:

1. A method of delivering an analgesic, the method comprising:
combining the analgesic with a DAG-PEG to produce a liposome suspension; and administering the suspension sublingually.

2. The method of claim 1, where the suspension is delivered by means of an aerosol spray.

3. The method of claim 1, where the analgesic is chosen from the group comprising naproxen, ibuprofen and acetaminophen ketoprofen, diclofenac, hydrocodone, morphine, fentanyl, hydromorphone, methadone, meperidine, oxycodone, and levorphanol.

4. The method of claim 2, further comprising providing the liposome suspension in the reservoir of an aerosol deliverer.

5. The method of claim 1, where the DAG-PEG has a P.sub.a between about 0.84 and 0.88 and a P.sub.v between about 0.88 and 0.93 and where P.sub.a is the packing parameter with respect to surface and P.sub.v is the packing parameter with respect to volume.

6. The method of claim 5, where said combining occurs at a temperature above the melting point of the DAG-PEG.

7. The method of claim 1, where the PEG chain of the DAG-PEG has a molecular weight between about 300 Daltons and 5000 Daltons.

8. The method of claim 1, where the DAG-PEG is selected from the group consisting of PEG-12 glycerol dioleate (GDO), PEG-12 glycerol dimyristate (GDM), PEG-23 glycerol dipalmitate (GDP), PEG-12 glycerol distearate (GDS), and PEG-23 GDS, where the number after “PEG” indicates the numbers of C₂H₄O subunits in the PEG chain.

9. The method of claim 1, where the melting point of the DAG-PEG is below about 40 degrees C., and where the acyl chains of the DAG-PEG are greater than or equal to 14 carbons in length.

10. An aerosol delivery system comprising:
an aerosol deliverer;
a reservoir;
a liposomal suspension contained in the reservoir, such liposomal suspension comprising an analgesic and a DAG-PEG.

11. The system of claim 10, where the analgesic is chosen from the group comprising naproxen, ibuprofen and acetaminophen, ketoprofen, diclofenac, hydrocodone, morphine, fentanyl, hydromorphone, methadone, meperidine, oxycodone, and levorphanol.

12. The system of claim 10, where the DAG-PEG has a P.sub.a between about 0.84 and 0.88 and a P.sub.v between about 0.88 and 0.93 and where P.sub.a is the packing parameter with respect to surface and P.sub.v is the packing parameter with respect to volume.

13. The system of claim 10, where the PEG chain of the DAG-PEG has a molecular weight between about 300 Daltons and 5000 Daltons.

14. The system of claim 10, where the DAG-PEG is selected from the group consisting of PEG-12 glycerol dioleate (GDO), PEG-12 glycerol dimyristate (GDM), PEG-23 glycerol dipalmitate (GDP), PEG-12 glycerol distearate (GDS), and PEG-23 GDS, where the number after “PEG” indicates the numbers of C₂H₄O subunits in the PEG chain.

15. The system of claim 10, where the melting point of the DAG-PEG is below about 40 degrees C., and where the acyl chains of the DAG-PEG are greater than or equal to 14 carbons in length.

16. A liposomal suspension comprising:
a DAG-PEG; and
an analgesic.

17. The suspension of claim 10, where the analgesic is chosen from the group comprising naproxen, ibuprofen and acetaminophen, ketoprofen, diclofenac, hydrocodone, morphine, fentanyl, hydromorphone, methadone, meperidine, oxycodone, and levorphanol.

18. The suspension of claim 10, where the DAG-PEG has a P.sub.a between about 0.84 and 0.88 and a P.sub.v between about 0.88 and 0.93 and where P.sub.a is the packing parameter with respect to surface and P.sub.v is the packing parameter with respect to volume.

19. The suspension of claim 10, where the PEG chain of the DAG-PEG has a molecular weight between about 300 Daltons and 5000 Daltons.

20. The suspension of claim 10, where the DAG-PEG is selected from the group consisting of PEG-12 glycerol dioleate (GDO), PEG-12 glycerol dimyristate (GDM), PEG-23 glycerol dipalmitate (GDP), PEG-12 glycerol distearate (GDS), and PEG-23 GDS, where the number after “PEG” indicates the numbers of C₂H₄O subunits in the PEG chain.

21. The suspension of claim 10, where the melting point of the DAG-PEG is below about 40 degrees C., and where the acyl chains of the DAG-PEG are greater than or equal to 14 carbons in length.
